(19) United States
(12) Patent Application Publication
(10) Pub. No.: US 2025/0360020 A1
(43) Pub. Date: Nov. 27, 2025

(54) Wearable Therapeutic Brace for the Treatment of Peyronie's Disease

(71) Applicants: Melanie D. Hatto, Cape Coral, FL (US); Alfred E. Cook, Rockport, TX (US)

(72) Inventors: Melanie D. Hatto, Cape Coral, FL (US); Alfred E. Cook, Rockport, TX (US)

(21) Appl. No.: 19/216,157

(22) Filed: May 22, 2025

Related U.S. Application Data
(60) Provisional application No. 63/650,639, filed on May 22, 2024.

(51) Int. Cl. A61F 5/41; A61F 5/37

(52) U.S. Cl. CPC A61F 5/41; A61F 5/3715; A61F 2005/411

(57) ABSTRACT

Therapeutic brace for holding and supporting the penis against the patient's body under tension to facilitate penile traction therapy. The therapeutic brace of this invention generally includes a main body support to be donned by a patient around the upper thick or abdomen and a penile support worn over the flaccid shaft of the penis. The main body support includes an adjustable overlie strap that partially overlies the length of the main body support. In use, the overlie strap is pulled tightly over penis and secured to the main body support to secure the penis under tension between the main body support and the overlie strap in the desired orientation and position against the patient's body.

This application claims the benefit of U.S. Provisional Patent Application, Ser. No. 63/650,639 filed May 22, 2024, the disclosure of which is hereby incorporated by reference.

This invention relates to a therapeutic brace for penile traction therapy in the treatment of Peyronie's Disease, and in particular, a wearable therapeutic brace for securely positioning and holding a penis to the body under tension.

### BACKGROUND AND SUMMARY OF THE INVENTION

Peyronie's disease is a condition where scar tissue, also referred to as plaque, forms on the tunica albuginea, the connective tissue that surrounds the corpus cavernosum and runs along the penis shaft. As scar tissue develops on the tunica albuginea, the penis may begin to curve, or change shape. Pain, erectile dysfunction (ED), hard lumps on one or more sides of the penis, curvature of the shaft with or without an erection, changes in the shape of the penis such as narrowing or shortening, sexual function impairment and depression are possible side effects of Peyronie's disease.

Penile traction therapy is the only reliable non-surgical treatment for Peyronie's disease. The penile traction therapy is a form of mechanotransduction and involves prolonged and low-load stretching of the flacid penis. The flaccid penis is physically manipulated and stretched to break up the plaque in the corpus cavernosum and then securely held under tension to facilitate remodeling of the tunica albuginea tissue. Penile traction therapy enhances penile flexibility and range of motion for those suffering from Peyronie's disease. Moreover, penile traction therapy is the only option for those that cannot afford the high cost of more invasive surgical and mechanical treatments, which are often painful and time-consuming and require the patient to be immobile for extended periods of time.

Therefore a need exists for a comfortable, convenient, affordable and discrete apparatus that supports proper penile alignment and holds the flaccid penis under tension to facilitate passive penile traction therapy in the treatment of Peyronie's disease and other erectile dysfunction.

### SUMMARY OF INVENTION

The therapeutic braces of this invention holds and supports the penis to the patient's body under tension to facilitate penile traction therapy. The therapeutic braces of this invention generally includes a main body support to be donned by a patient around the upper thick or abdomen and a penile support worn over the flaccid shaft of the penis. The main body support includes an adjustable overlay strap that partially overlies the length of the main body support. In use, the overlay strap is pulled tightly over the penis and secured to the main body support to secure the penis under tension between the main body support and the overlie strap in the desired orientation and position against the patient's body. The brace does not physically stretch or elongate the penis, but positions and holds the penis flaccid under tension against the body to facilitate penile traction therapy. The brace enhances patient comfort and can be discretely worn under clothing during treatment.

The above described features and advantages, as well as others, will become more readily apparent to those of ordinary skill in the art by reference to the following detailed description and accompanying drawings.

### BRIEF DESCRIPTION OF THE DRAWINGS

The present invention may take form in various system and method components and arrangement of system and method components. The drawings are only for purposes of illustrating exemplary embodiments and are not to be construed as limiting the invention.

FIG. 1 is a perspective view of an exemplary embodiment of the therapeutic support of this invention donned on the thigh of a user;

FIG. 2 is a perspective view of the penile support of the therapeutic brace of FIG. 1 donned by a user;

FIG. 3 is a perspective view of the main body support of the therapeutic brace of FIG. 1 shown laid flat;

FIG. 4 is a top view of the penile support of the therapeutic brace of FIG. 1 shown laid flat;

FIG. 5 is a top view of another exemplary embodiment of the penile support used in support therapeutic support of this invention, shown laid flat;

FIG. 6 is a perspective view of the penile wrap of FIG. 5 shown wrapped over itself;

FIG. 7 is a perspective view of a main body sleeve used in another exemplary embodiment of the therapeutic brace of this invention;

FIG. 8 is a perspective view of a penile sleeve used in another exemplary embodiment of the therapeutic brace of this invention; and

FIG. 9 is a perspective view of the therapeutic support of FIG. 1 shown with the main body wrap donned around the abdomen of a user.

### DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical, structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

The therapeutic braces of this invention holds and supports the penis to the patient's body under tension to facilitate penile traction therapy. Each embodiment of the therapeutic brace of this invention generally includes a main body support to be donned by a patient around the upper thick or abdomen and a penile support worn over the flaccid shaft of the penis. The elasticity of the supports allows the braces to be easily donned by the patient and the penis securely held against the body under tension to create the necessary stretch and elongation to the scar tissue. The braces do not physically stretch or elongate the penis, but position and hold the penis flaccid under tension against the body to facilitate penile traction therapy. The braces enhance patient comfort and can be discretely worn under clothing during treatment.

Certain embodiments of the therapeutic brace may employ “wrap” style body and penile supports that are elastics bands that wrap around and adhere to themselves to tightly and securely don the support about the thigh, abdomen or penis. Other embodiments of the therapeutic brace may employ a “sleeve style body and penile supports that are continuous cylindrical elastic loops or sleeves that are stretched and pulled over the thighs, abdomen and penis.

The main body and penile supports are made or fashioned from thin pliable elastic fabrics, and may employ any combination of elastomeric or non-elastomeric nylons, polyesters, knit and/or layered fabrics or closed cell foams, such as Neoprene. The fabrics have a soft pliable, non-absorbent cloth hand on both outer and inner facing surfaces suitable for contacting the patient's skin. The fabrics are also light weight and thin to allow the braces to be discretely worn under the patient's normal clothing. The fabrics may also include surface treatments, fabric sections and patches of non-slip materials to assist securely supporting the penis to the body within the brace.

Fabrics are also selected to allow the use of conventional hook and loop fastening elements to be used to connect together overlapped ends of “wrap” style supports, as well as to interconnect the separate penile and main body supports to one another. The hook and loop fasteners may take the form of the base fabrics having inner and outer surfaces with opposed hook and loop pile or separate hook and loop patches sewn directly thereto, or any combination of such fabric features. In alternative embodiments, such support connections may be facilitated by other conventional methods and means, such as buckles, buttons, coupling fasteners, laces, strapping and snaps.

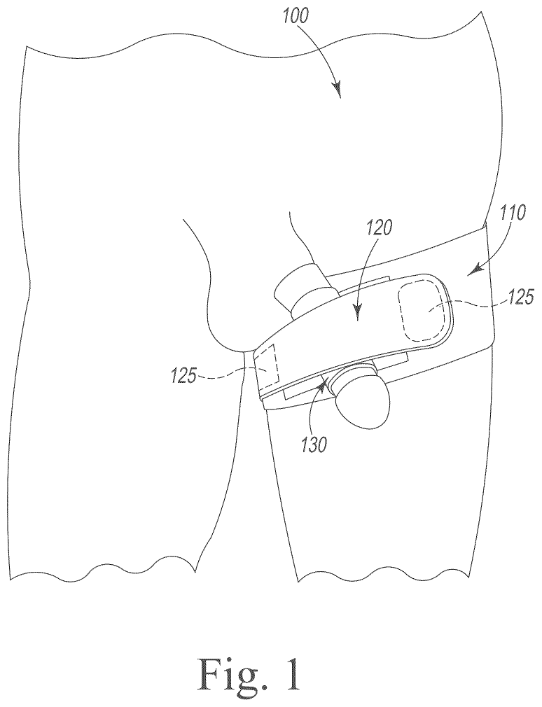

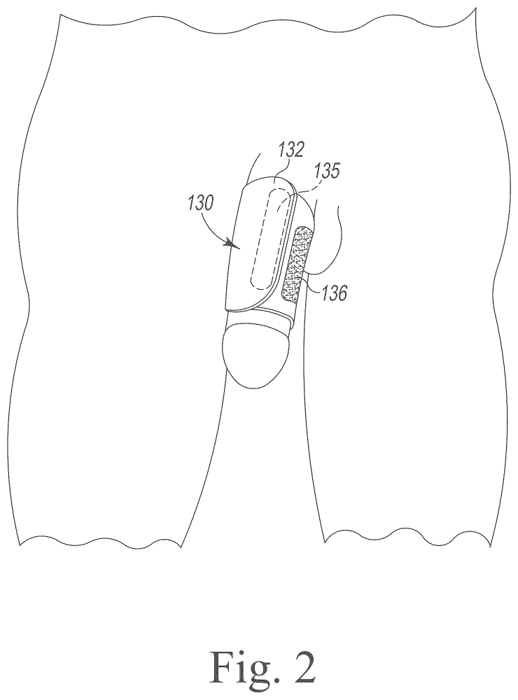

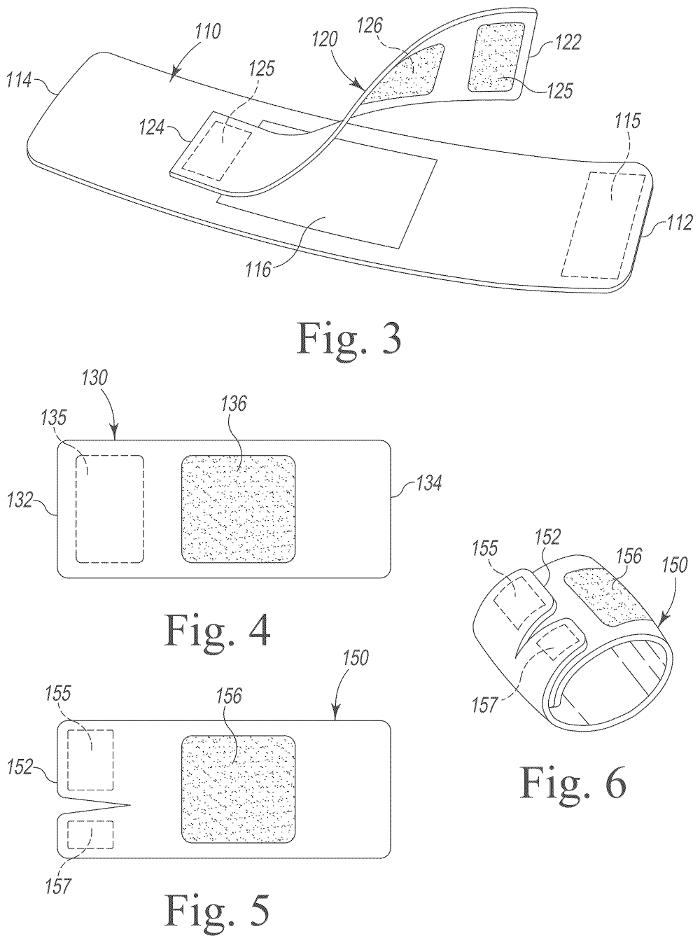

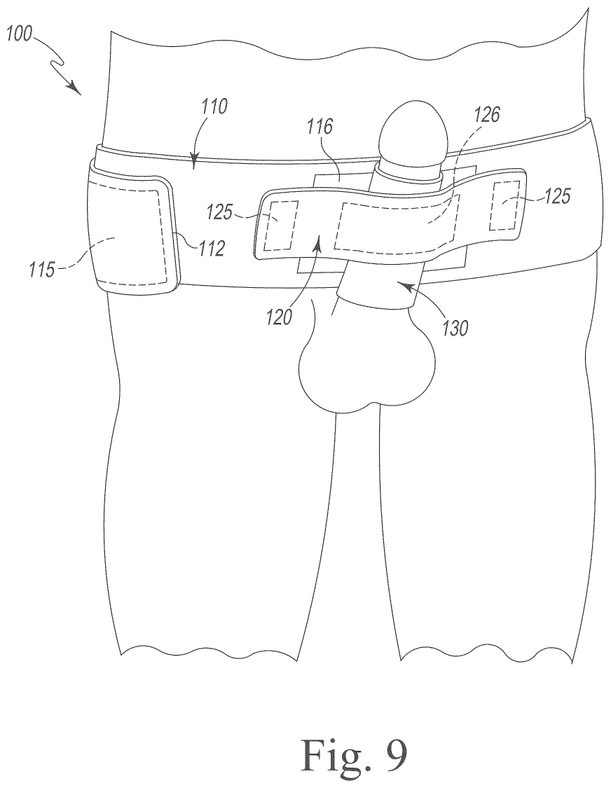

Referring now to the drawings, FIGS. 1-4 and 9 illustrate an exemplary embodiment of the wearable therapeutic brace of this invention, which is designated generally as reference numeral 100. Therapeutic brace 100 includes a “wrap” style main body support 110 and a wrap style penial support or wrap 130. Main body support 110 is an elongated elastic band dimensioned to wrap around the patient's thigh (FIG. 1) or the abdomen (FIG. 9). Main body support 110 has two opposed ends 112 and 114 that overlap one another when donned. Wrap end 114 has a hook and loop fastener 115 on its inner surface that allows end 114 to overlap end 112 and detachably adhere to the outer surface of main body support 110. The outer surface of main body wrap 110 also includes a non-slip feature 116, which may take the form of a sewn on patch or fabric treatment.

Main body support 110 also includes an adjustable overlay strap 120. Again, overlay strap 120 is an elongated elastic band that partially overlies the length of main body support 110. Overlay strap 120 has two opposed ends 122 and 124 that are detachably affixed to main body support 110 by hook and loop fasteners 125 affixed to the inner surface of the overlay strap. In certain embodiments of the overlay strap, one end may be permanently affixed or sewn to the main body support allowing the other free end to be detachable from the main body support. The inner surface of overlay strap 120 also includes a hook and loop patch 126.

As shown best in FIGS. 1, 2 and 4, penile support 130 is also an elongated elastic band dimensioned to wrap around and overlap the shaft of the patient's penis. Penile support 130 has two opposed ends 132 and 134 that overlap one another when donned. Support end 132 has a hook and loop fastener 135 on its inner surface that allows end 132 to overlap end 134 and detachably adhere to the outer surface of penile support 130. The outer surface of penile support 130 also includes a hook and loop fastener 136 adapted to engage and secure connected penile support 130 between main body support 110 and overlay strap 120.

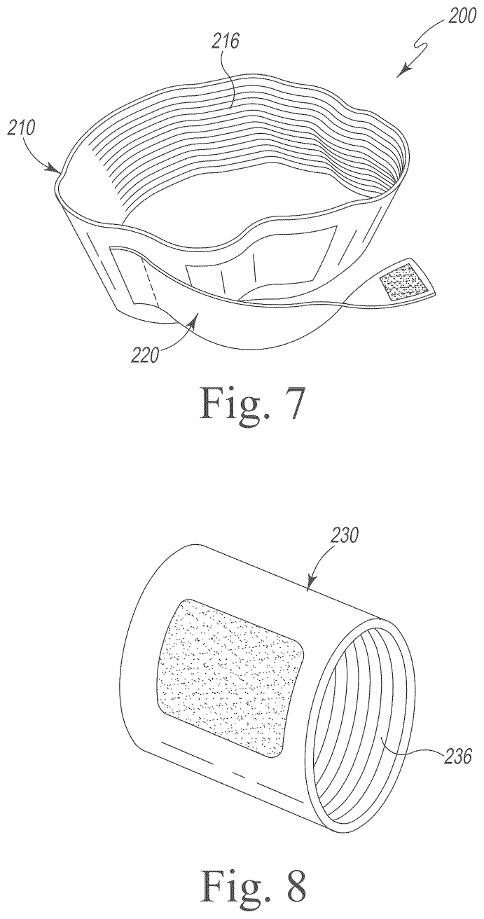

FIGS. 5 and 6 illustrate an alternative exemplary embodiment of a “wrap” style penile support of this invention, which is designated generally as reference numeral 150. Penile support 150 is identical to penile support 130 in construction and function, except that one end 152 is bifurcated and has two separate hook and loop fasteners 155 and 157. The bifurcated ends and separate hook and loop fasteners, help facilitate the donning and tightening of penile support 150 around the shaft of the penis. Again, the outer surface of penile support 150 also includes a hook and loop fastener 156 adapted to engage and secure connected penile support 150 between main body support 110 and overlay strap 120.

In use, main body support 110 and penile support 130 are first donned by the patient. Main body support 110 is wrapped around either the patient's thigh (FIG. 1) or the patient's abdomen (FIG. 9). It should be noted that brace 100 allows the penis to be securely held under tension in multiple positions and orientation for penile traction therapy. The main body support 110 can be donned on either leg, as well as around the abdomen.

Generally, main body support 110 is donned around the thigh to sit high on the thigh adjacent and underneath the scrotum, or donned around the patient's abdomen to sit low on the patient's hips. Opposed ends 112 and 114 of main body support 110 are secured by hook and loop fastener 115 adhering to the outer surface of the overlapped end with the support stretched tightly around the thigh or abdomen to hold the support in place. Penile support 130 is wrapped around the shaft of the flaccid penis (FIGS. 1, 2 and 9). Opposed ends 132 and 134 of penile support 130 are secured by hook and loop fastener 135 adhering to the outer surface of the overlapped ends with the support stretched comfortably around the penile shaft to hold the support in place along the shaft.

Once main body support 110 and penile support 130 are donned, overlay strap 120 is attached to main body support 110 at one strap end 122 so that the opposed free end 124 is spaced from the main body support. The patient or a qualified medical professional manually stretches and manipulates the penile shaft to position and hold the penis under tension against non-slip feature 116 across main body support 110. The free end 124 of overlie strap 120 is pulled tightly over the penile shaft and secured to main body support 110 to secure the penis under tension between the main body support 110 and loverlie strap 120 in the desired orientation and position for penile traction therapy. When overlay strap 120 is attached to main body support 110, hook and loop fasteners 136 of penile support 130 engages the hook and loop fastener 126 of overlay strap 120 to interconnect and hold the penile strap to the main body support. The use of the overlay strap and the various hook and loop fasteners allow the tension, orientation and position of the penis to be easily adjusted by the patient or medical professionals to aid in comfort and treatment. Non-slip feature 116 aids in securely positioning and holding the penis between the main body support 110 and overlay strap 120 during use.

FIGS. 7 and 8 illustrate a second exemplary embodiment of the wearable therapeutic brace of this invention, which is designated generally as reference numeral 200. Therapeutic brace 200 includes a “sleeve” style main body support 210 and a “sleeve” style penial support or wrap 230. Therapeutic brace 200 is similar in construction, features and function to brace 100 above, except that main body support 210 and penile support 230 are pulled onto rather than being wrapped around the leg and penis. Generally, main body support 210 and penile support 230 are constructed of elastic bands sewn end to end to form a continuous cylindrical sleeve that stretches to conform around the leg, abdomen and penile shaft. Both main body support 210 and penile support 230 have a non-slip treatment on their inner surfaces 216 and 236, which helps hold the supports in position about the thigh and penis. Again, main body support 210 includes overlay strap 220 that partially overlies the length of main body support 210 to secure penile wrap 230.

It should be noted that in certain applications of the therapeutic brace of this invention, the main body support may be used without the use of a penile support. In such applications, the overlay strap may be sufficient to hold the penis under tension in the desired orientation and position for penile traction therapy without employing a penile support, which certain patient's may find more comfortable. In addition, other embodiments of the therapeutic brace of this invention may employ a ridged component, such as an inserted plastic stay to help reinforce, orient and position the penis with respect to the main body support.

It should be apparent from the foregoing that an invention having significant advantages has been provided. While the invention is shown in only a few of its forms, it is not just limited but is susceptible to various changes and modifications without departing from the spirit thereof. The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

I claim:

1. A therapeutic brace for penile traction therapy in the treatment of Peyronie's Disease, the brace comprising:
a main body support adapted to be donned around the body of a patient,
the main body support includes an elongated strap connected to and overlying the main body support to restrictively hold the penis of the patient against the body of the patient and across the main body support between the strap and the main body support under tension during penile traction therapy.

2 The therapeutic brace of claim 1 and a penile support adapted to be donned around the penile shaft of the patient and interconnected to the main body support between the main body support and the strap.

3. The therapeutic brace of claim 2 wherein the penile support is detachably connected between the main body support and the strap during penile traction therapy.

4. The therapeutic brace of claim 3 wherein the penile support includes a fastener for detachably engaging one of the main body support and the strap.

5. The therapeutic brace of claim 1 wherein the main body wrap includes a non-slip feature.

6. The therapeutic brace of claim 5 wherein the non-slip feature partially covers the outer surface of the main body support.

7. The therapeutic brace of claim 1 wherein the main body support is an elongated elastic band having opposed ends.

8. The therapeutic brace of claim 7 wherein the opposed ends of the main body support are detachably connected to allow the main body support to be donned around the body of the patient.

9. The therapeutic brace of claim 2 wherein the penile support is an elongated elastic band having opposed ends.

10. The therapeutic brace of claim 9 wherein the opposed ends of the main body support are detachably connected to allow the main body support to be donned around the body of the patient.

11. The therapeutic brace of claim 1 wherein the main body support is an elastic sleeve adapted to be pulled over the body of the patient.

12. The therapeutic brace of claim 11 wherein the main body support includes a non-slip feature on the inner surface thereof.

13. The therapeutic brace of claim 2 wherein the main body support is an elastic sleeve adapted to be pulled over the body of the patient.

14. The therapeutic brace of claim 13 wherein the main body support includes a non-slip feature on the inner surface thereof.

15. The therapeutic brace of claim 1 wherein the main body support is an elongated elastic band configured to wrap around the upper thigh of the patient.

16. The therapeutic brace of claim 1 wherein the main body support is an elongated elastic band configured to wrap around the lower abdomen of the patient.

17. A therapeutic brace for penile traction therapy in the treatment of Peyronie's Disease, the brace comprising:
a main body support adapted to be donned around the body of a patient; and
a penile support adapted to be donned around the penile shaft of the patient,
the penile support is detachably connected to the main body support to hold the penis of the patient in a desired position and orientation against the body of the patient under tension during penile traction therapy,
the main body includes an elongated strap connected to and overlying the main body support to restrictively hold the penile support against the body of the patient and across the main body support between the strap and the main body support.
